(19) United States
(12) Patent Application Publication
(10) Pub. No.: US 2018/0105784 A1
(43) Pub. Date: Apr. 19, 2018

(54) CULTURE MEDIUM EXCHANGE UNIT

(71) Applicant: TAKASAGO ELECTRIC, INC., Nagoya-shi, Aichi (JP)

(72) Inventors: Naoya Asai, Nagoya-shi, Aichi (JP); Ken Naito, Nagoya-shi, Aichi (JP); Akihito Takatsuka, Nagoya-shi, Aichi (JP); Tomoki Koyama, Nagoya-shi, Aichi (JP); Tomohiro Sakashita, Nagoya-shi, Aichi (JP); Manaho Asahi, Nagoya-shi, Aichi (JP)

(21) Appl. No.: 15/561,697

(22) PCT Filed: Mar. 30, 2015
(86) PCT No.: PCT/JP2015/059971

§ 371 Date: Sep. 26, 2017

(51) Int. Cl. C12M 1/00; C12M 3/00; C12M 1/36

(52) U.S. Cl. CPC C12M 23/50; C12M 41/48; C12M 23/48

(57) ABSTRACT

Inside a case (1) of a culture medium exchange unit, a well plate (3) with wells (4) is disposed, and cells and a culture medium are placed into each of the wells (4) of the well plate (3) and cell culture is performed, and after a predetermined period of time, the culture medium inside each well (4) is exchanged. The well plate (3), a supply liquid contact part (8) of a supply pump (6), a supply tube (9), supply nozzles (10), discharge nozzles (17), discharge tubes (15), and discharge liquid contact parts (14) are disposed so as to be removable from the case (1) and replaceable.

### TECHNICAL FIELD

The present invention relates to a culture medium exchange unit to be used for cell culture (including culture of body tissue, etc.).

### BACKGROUND ART

In recent years, research to achieve practical use of iPS cells and ES cells, etc., has been advanced, and accordingly, research to provide regenerative medicine by culturing cells and body tissue and transplanting the culture into a human has been actively performed.

In the culturing step of performing the cell culture in this type of research, for example, cells and body tissue are contained together with a culture medium (liquid culture medium) in a well plate as a culture vessel, provided with a plurality of wells, and the cells and body tissue are cultured, and in this case, processing to periodically exchange the culture medium inside the wells of the well plate is performed.

As a culture medium exchange device that performs this kind of culture medium exchange, conventionally, a culture medium exchange device that takes out a well plate from an incubator and automatically exchanges a culture medium in each well of the well plate by using a culture medium exchange robot, has been proposed in Patent Literature 1 listed below. In this culture medium exchange device, a culture medium exchange robot automatically performs operations of taking out a well plate from the inside of an incubator and taking out a culture medium from the inside of each well, and then supplying a new culture medium into each well, and returning the well plate in which culture medium exchange was performed into the incubator again.

### CITATION LIST

### Patent Literatures

Patent Literature 1: JP 2002-262856 A

Patent Literature 2: JP 2002-153256 A

### SUMMARY OF INVENTION

### Technical Problem

However, in the culture medium exchange device that places a well plate in and out from an incubator and exchanges a culture medium by using an exchange robot as described above, inevitably, an exchange robot that three-dimensionally moves needs to be installed, so that the device becomes very large in size, and the manufacturing cost of the culture medium exchange device is increased. Therefore, the culture medium exchange device becomes expensive and it is difficult to easily introduce this device in many research institutions, and thus, it has been demanded to realize an easily usable and inexpensive culture medium exchange device that can be introduced in a small-scale research institution.

On the other hand, a comparatively compact device that exchanges a culture medium inside a cell culture vessel without using an exchange robot is proposed in Patent Literature 2 listed above. This culture medium exchange device supplies a culture medium by using a liquid feed pump into a cell culture vessel fixedly installed, and after a predetermined period of time, discharges the culture medium from the cell culture vessel by using the same liquid feed pump, and then supplies a new culture medium into the cell culture vessel from a culture medium supply vessel by the liquid feed pump, whereby exchanging the culture medium.

However, although this culture medium exchange device performs culture medium exchange without using an exchange robot and accordingly, the device can be configured comparatively compact, and to one liquid feed pump, one cell culture vessel, a culture medium containing vessel, and a waste culture medium vessel are connected via piping and connectors in each one system, so that in the case of exchanging a culture medium in a well plate with a large number of wells, the number of liquid feed pumps and the number of vessels become very large, and eventually, it still has a problem of an increase in size.

In addition, in the culture medium exchange device, to prevent contamination, liquid contact parts of the liquid feed pump and piping used once for cell culture need to be completely cleaned or disposed of, however, this conventional culture medium exchange device is structured by fixedly connecting a cell culture vessel, a culture medium containing vessel, and a waste culture medium vessel via piping and connectors, so that liquid contact parts of the used liquid feed pump and piping cannot be removed and cleaned or disposed of. Therefore, it has been demanded to realize a culture medium exchange unit in which liquid contact parts of pumps, piping, and valves, etc., can be easily replaced and cleaned or disposed of.

The present invention solves the problem described above, and an object thereof is to provide a culture medium exchange unit for cell culture which has a simple structure and can be inexpensively manufactured, and in which liquid contact parts can be easily cleaned or disposed of.

### Solution to Problem

A culture medium exchange unit according to the present invention in which a well plate with wells is disposed, and which performs cell culture by placing cells and a culture medium into each well of the well plate, and after a predetermined period of time, exchanges the culture medium in each well, includes:

a case including an accommodation chamber that accommodates the well plate in a manner enabling the well plate to be taken out;

an opening and closing lid provided to cover the accommodation chamber of the case and be openable and closable;

a supply pump that supplies a culture medium into each of the wells of the well plate;

a discharge pump that discharges a used culture medium from the wells;

supply nozzles that supply a culture medium fed from the supply pump through supply tubes into the wells; and

discharge nozzles that discharge a culture medium from the inside of the wells in response to an operation of the discharge pump, wherein

the well plate, the supply pump, the discharge pump, the supply nozzles, and the discharge nozzles are accommodated in the case,

to the supply pump, a supply liquid contact part serving as a culture medium flow path and a supply pump drive part that operates to feed a culture medium into the supply liquid contact part by driving a drive part are joined in a separable manner,

to the discharge pump, a discharge liquid contact part serving as a culture medium flow path and a discharge pump drive part that operates to discharge a culture medium from the inside of the discharge liquid contact part by driving a drive part are joined in separable manner,

supply tubes are connected to the supply nozzles, discharge tubes are connected to the discharge nozzles, a culture medium is supplied to the supply nozzles from the supply liquid contact part of the supply pump through the supply tubes, and a used culture medium is discharged from the discharge nozzles through the discharge tubes and the discharge liquid contact part, and

the well plate, the supply liquid contact part of the supply pump, the supply tubes, the supply nozzles, the discharge nozzles, the discharge tubes, and the discharge liquid contact part are disposed to be removable from the case and replaceable.

According to the present invention, the supply pump, the supply tubes, the supply nozzles, the discharge tubes, and the discharge pump for a culture medium are accommodated in the same case that accommodates wells having a well plate, and accordingly, the case can be formed compact, so that a user can place a culture medium exchange unit in and out from an incubator as it is in a small-sized case, and use it conveniently. After use, the well plate, the supply liquid contact part of the supply pump, the supply tubes, the supply nozzles the discharge nozzles, the discharge tubes, and the discharge liquid contact part are taken out from the case, and can be cleaned or disposed of, so that the unit can be repeatedly used for cell culture at a low cost while preventing contamination of cells.

Here, it is preferable that flow paths to be connected to the supply liquid contact part and the discharge liquid contact part are integrated as a flow path manifold, and the flow path manifold is formed in a manner enabling it to be taken out and replaced. Accordingly, when cleaning or disposing of the liquid contact parts, the liquid contact parts can be very easily taken out and replaced.

Here, it is preferable that in the flow path manifold described above, to the flow paths between the supply pump and the supply nozzles, on-off valves are respectively connected. Accordingly, a culture medium fed from one supply pump can be supplied by way of on-off valves connected to the respective flow paths, and a predetermined amount of the culture medium can be uniformly supplied into each well.

In addition, the supply pump can consist of a peristaltic pump, and the supply liquid contact part can be configured to have an arc-shaped flexible flow path to be pressed by a rotor. This makes it easy to clean or dispose of the liquid contact part.

In addition, the unit can also be configured so that the supply pump consists of a piezoelectric diaphragm type piezo pump, and the supply liquid contact part has a diaphragm chamber.

### Effects of the Invention

According to the culture medium exchange unit of the present invention, the structure is simple and can be manufactured inexpensively, and liquid contact parts can be easily cleaned or disposed of.

### BRIEF DESCRIPTION OF THE DRAWINGS

FIG. 1 is a schematic entire configuration diagram of a culture medium exchange unit showing an embodiment of the present invention.

FIG. 2 is an exploded perspective view of the culture medium exchange unit.

FIG. 3 is an exploded perspective view of the culture medium exchange unit.

FIG. 4 is an exploded perspective view of the culture medium exchange unit viewed from a diagonally lower side.

FIG. 5 is an exploded front view of the culture medium exchange unit.

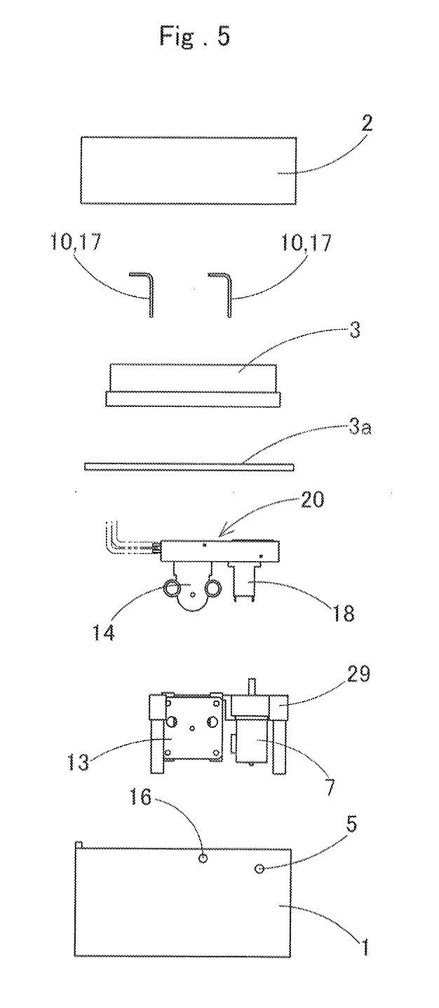

FIG. 6 is an exploded right side view of the culture medium exchange unit.

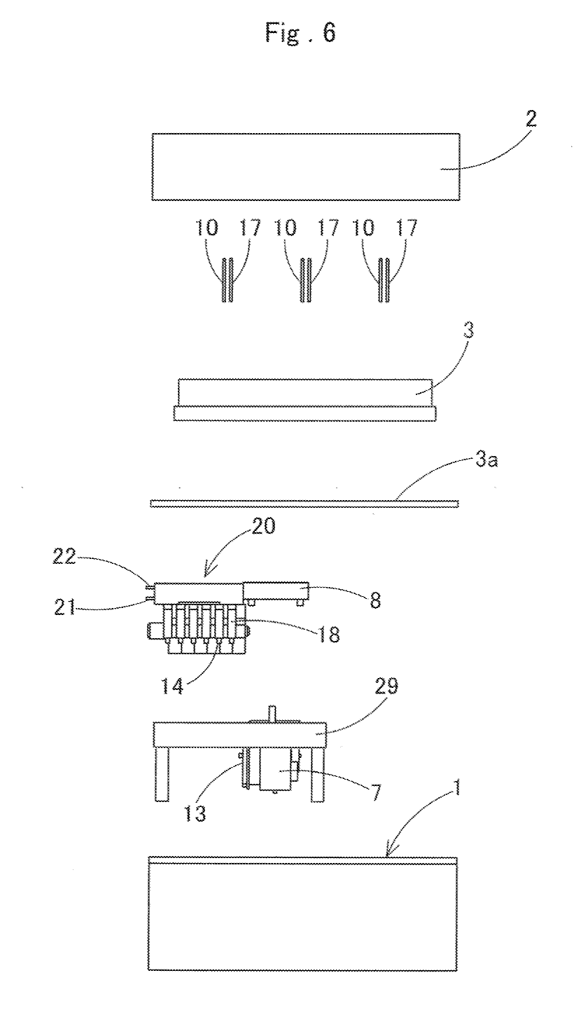

FIG. 7 is an exploded back view of the culture medium exchange unit.

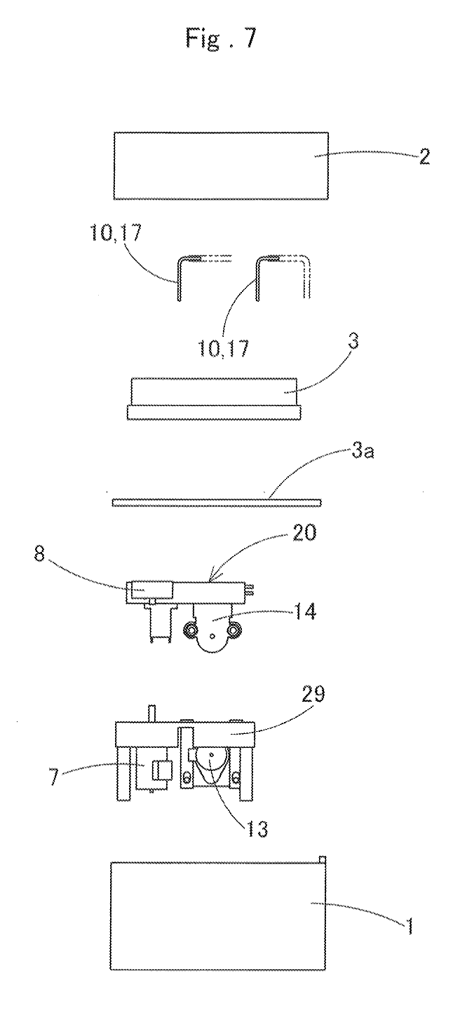

FIG. 8(a) is a perspective view, FIG. 8(b) is a plan view, and FIG. 8(c) is a bottom view of a flow path manifold.

FIG. 9 is a perspective view showing connections of tubes and nozzles of the flow path manifold.

FIG. 10(a) is a perspective view of a discharge pump, and FIG. 10(b) is a perspective view of the discharge pump viewed from another angle.

FIG. 11(a) is an exploded perspective view of the discharge pump, FIG. 11(b) is an exploded perspective view of the discharge pump viewed from another angle, FIG. 11(c) is an exploded front view of the same, and FIG. 11(d) is an exploded bottom view of the same.

FIG. 12 is a schematic sectional view of a discharge liquid contact part of the discharge pump.

FIG. 13(a) is a plan view of a supply pump, FIG. 13(b) is a perspective view showing a disassembled state of a supply pump drive part and a supply liquid contact part, and FIG. 13(c) is a perspective view of the same from another angle.

FIG. 14 is a sectional explanatory view showing the inside of a case.

### DESCRIPTION OF EMBODIMENT

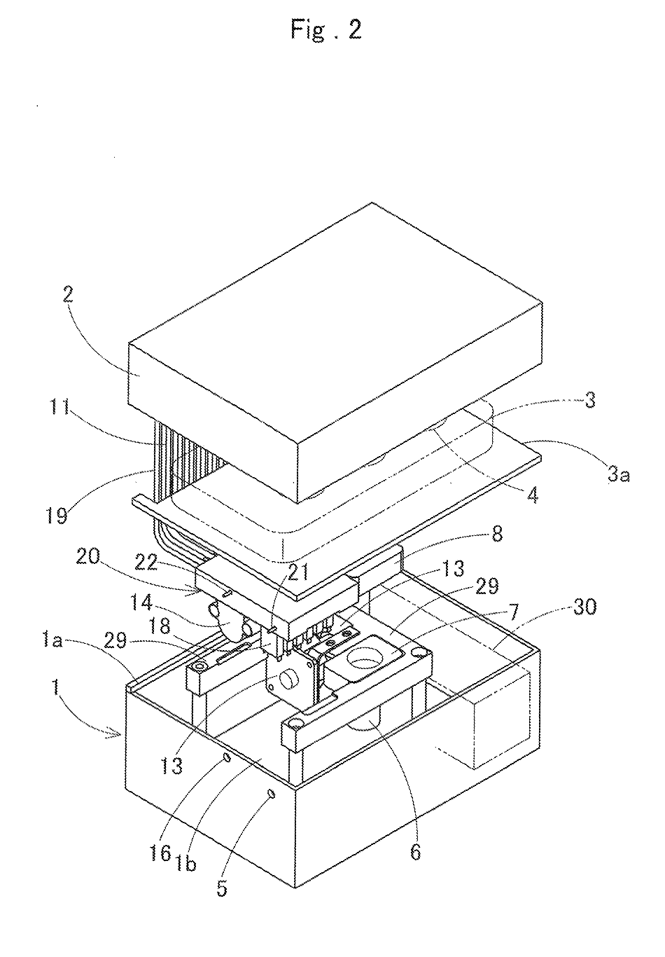

Hereinafter, an embodiment of the present invention is described with reference to the drawings. This culture medium exchange unit includes, as shown in FIG. 2, a well plate (multi-well plate) 3 with a plurality of (here, six) wells 4 disposed inside a case 1, and is configured so that cells and a culture medium are placed in each well 4 of the well plate 3, the well plate is placed in an incubator not shown in the figure and the temperature and the humidity are controlled, and during cell culture inside the wells 4, after elapse of a period of time set in advance, an old culture medium is discharged from the wells 4, and a fresh culture medium is supplied into the wells 4, whereby automatic exchange of the culture medium is performed.

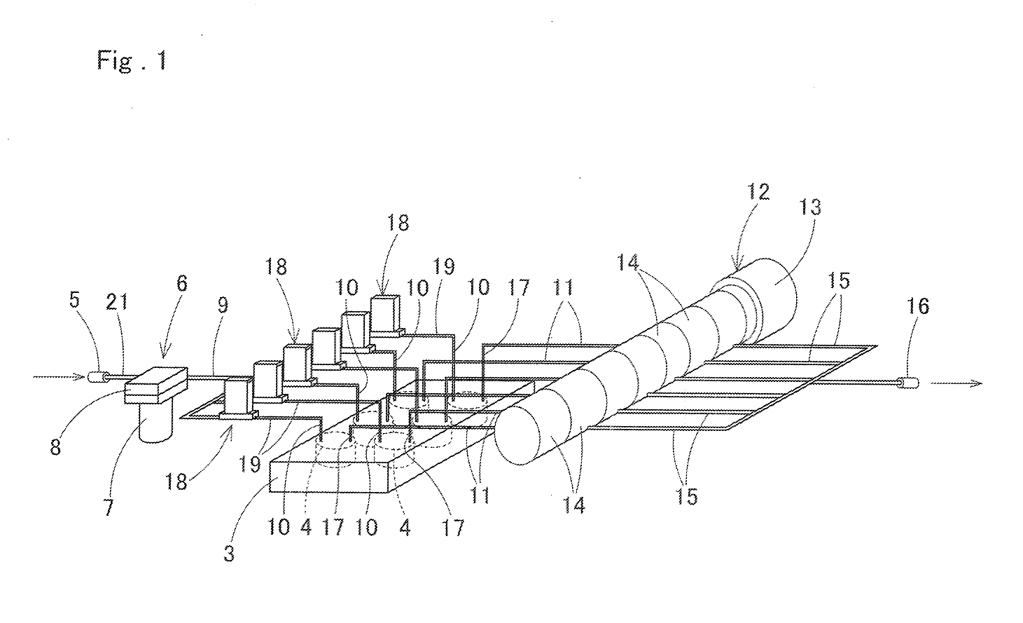

As shown in FIG. 1 and FIG. 2, generally, the culture medium exchange unit includes the case 1 having an accommodation chamber 1b that accommodates the well plate 3 in a manner enabling the well plate to be taken out, an opening and closing lid 2 provided to cover the accommodation chamber 1b of the case 1 and be openable and closable, a supply pump 6 that supplies a culture medium into each well 4 of the well plate 3, a discharge pump 12 that discharges a used old culture medium from the wells 4, a plurality of supply nozzles 10 that supply a culture medium fed from the supply pump 6 through supply tubes 9 and 19 into each of the wells 4, discharge nozzles 17 that discharge a culture medium from the inside of each well 4 in response to an operation of the discharge pump 12, and a controller 30 that controls the supply pump 6 and the discharge pump 12. Inside the small-sized case 1 formed compact, the accommodation chamber 1b is formed, and inside the accommodation chamber 1b, the well plate 3, the supply pump 6, the discharge pump 12, the supply nozzles 10, the discharge nozzles 17, and the controller 30 are accommodated as shown in FIG. 2.

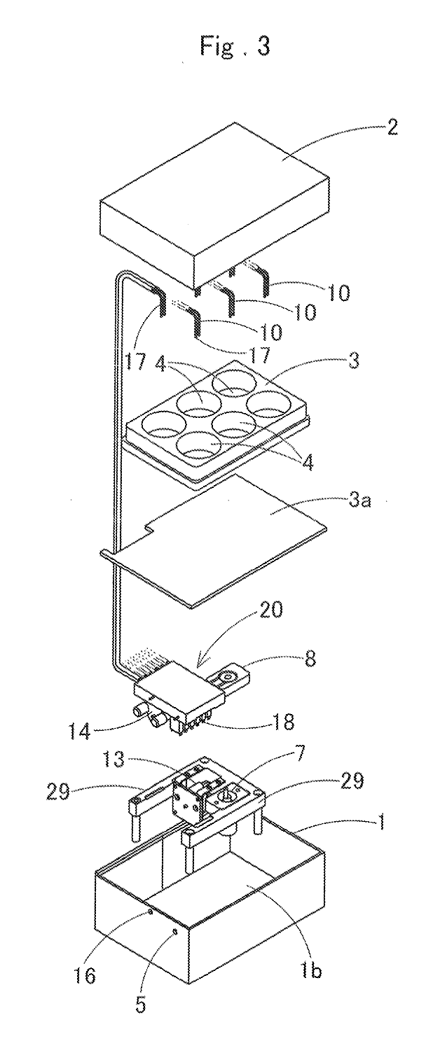

The case 1 is formed into, as shown in FIG. 2, a rectangular parallelepiped box shape, and in an upper portion of the accommodation chamber 1b inside the case 1, the well plate 3 is accommodated on a support plate 3a. Further, in a lower portion of the accommodation chamber 1b, the controller 30 is accommodated, and further, as shown in FIG. 3, a supply pump drive part 7 that drives the supply pump 6 and a discharge pump drive part 13 that drives the discharge pump 12 are fixed to an attaching frame 29 and accommodated.

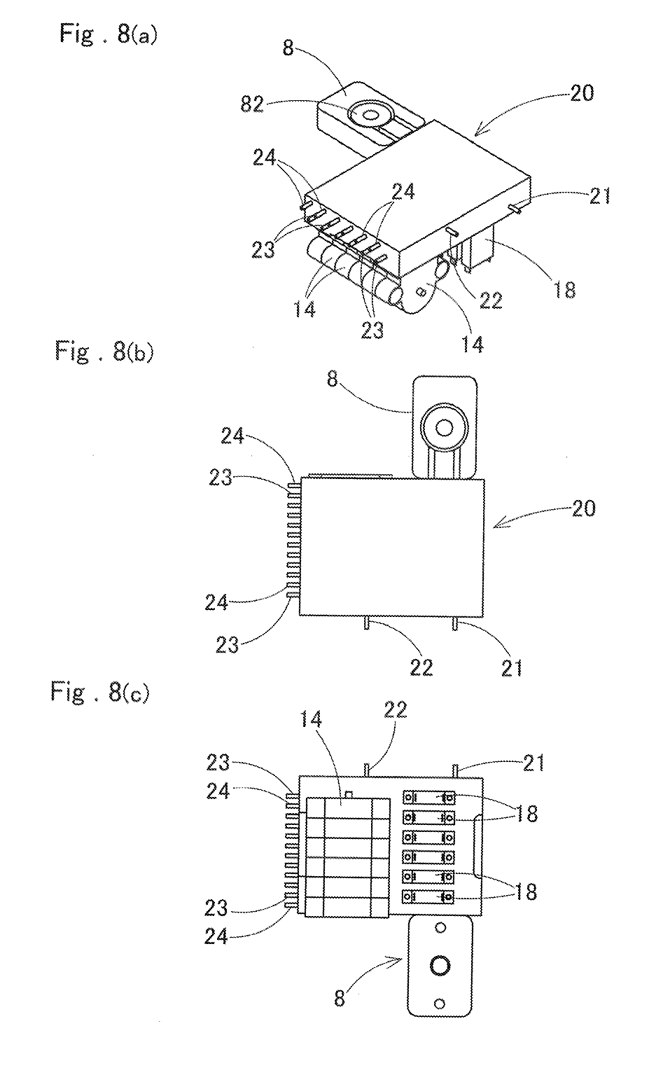

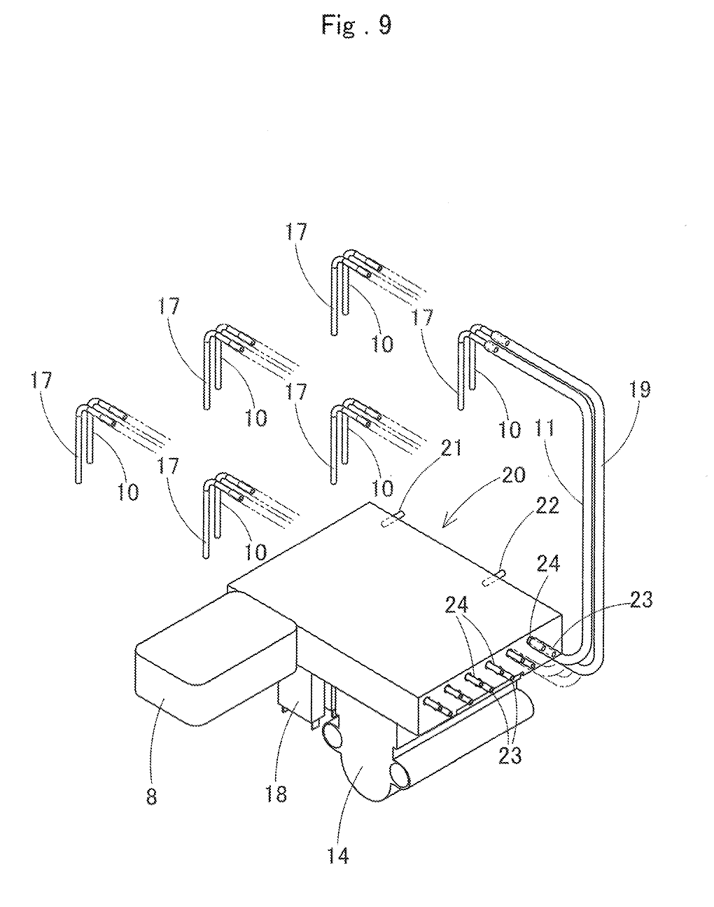

To an upper portion of the supply pump drive part 7 and the discharge pump drive part 13, a flow path manifold 20 shown in FIG. 8 and FIG. 9 is attached in a separable manner. Further, the support plate 3a is attached to an upper portion of the accommodation chamber 1b of the case 1 in a horizontal lateral direction, and the well plate 3 is placed on the support plate 3a and accommodated in a manner enabling the well plate to be taken out and replaced. At present, many kinds of well plates have been manufactured and used for cell culture, and they can be used as they are. The well plate 3 shown in the drawings is a micro plate provided with six wells 4, and a multi-well plate with an arbitrary size having an arbitrary number of wells, such as 1, 12, or 24 wells can also be used.

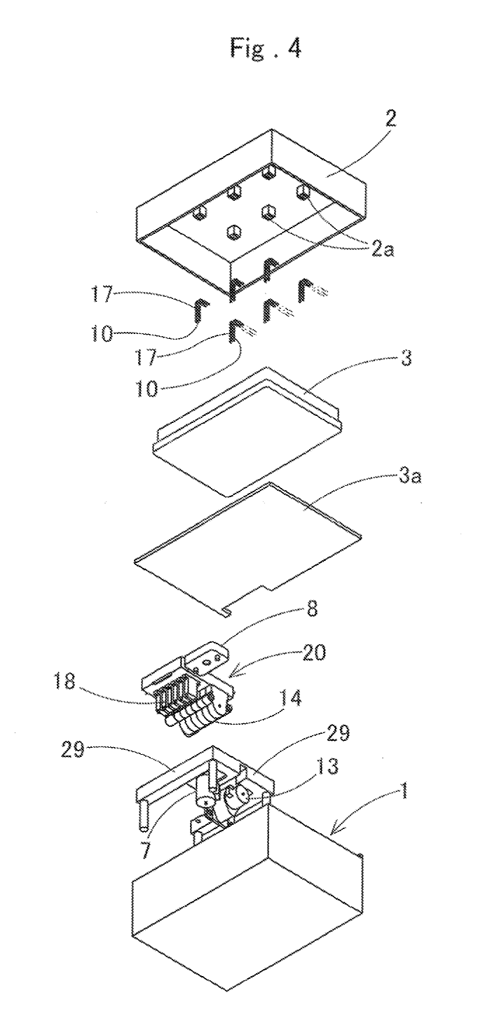

Above the well plate 3, as shown in FIG. 2, an upper portion of the case 1 is closed in an openable manner by the opening and closing lid 2. Although the opening and closing lid 2 is shown in FIG. 2 in a separated state, a side of a lower portion of the opening and closing lid 2 is connected to the upper portion of the case 1 via a hinge 1a, and closes and covers the upper side of the well plate 3 in an openable manner via the hinge 1a. As shown in FIG. 4, inside the opening and closing lid 2, nozzle attaching portions 2a are provided at six points on the upper sides of the respective wells 4. To these nozzle attaching portions 2a, the supply nozzles 10 that supply a culture medium into the wells 4 and discharge nozzles 17 that discharge a culture medium from the wells 4 are respectively attached, and supported.

Tip end positions of the supply nozzles 10 and the discharge nozzles 17 can be adjusted at the nozzle attaching portions 2a, and the tip ends of the supply nozzles 10 are positioned higher than the tip ends of the discharge nozzles 17, and the tip ends of the discharge nozzles 17 are positioned lower than the tip ends of the supply nozzles 10. Accordingly, the tip ends of the supply nozzles 10 can be prevented from coming into contact with a culture medium, and contamination can be prevented.

As shown in FIG. 9, the respective supply nozzles 10 of six systems in the case 1 are formed to be shorter than the respective discharge nozzles 17, and the respective discharge nozzles 17 of six systems are formed to be longer than the supply nozzles 10, and thus, height positions of their tip ends can be adjusted. Accordingly, when the supply nozzles 10 are inserted into the wells 4, the tip ends of the supply nozzles 10 are positioned higher than the tip end portions of the discharge nozzles 17, and the tip ends of the discharge nozzles 17 are positioned lower than the tip ends of the supply nozzles 10, and therefore, culture medium amounts in the wells 4 can be adjusted. In actual cell culture, at the time of culture medium exchange, half of the old culture medium in each well 4 is discharged while the other half is left to perform half-amount exchange.

As shown in FIG. 9, six supply nozzles 10 are respectively connected to supply ports 23 of the flow path manifold 20 via the supply tubes 19, and six discharge nozzles 17 are respectively connected to discharge ports 24 of the flow path manifold 20 via discharge tubes 11. As shown in FIG. 8 and FIG. 9, the flow path manifold 20 introduces a culture medium through a main inlet port 21 provided on the front surface, and feeds the culture medium from six supply ports 23 through a supply liquid contact part 8 and branch flow paths of the supply pump 6 formed inside the flow path manifold.

Further, in the flow path manifold 20, discharge liquid contact parts 14 of the discharge pump 12 are provided, and six discharge ports 24 on a side surface are provided, and on the front surface of the flow path manifold 20, a main outlet port 22 is provided. Accordingly, through the six discharge ports 24 on the left side surface of the flow path manifold 20, a culture medium is suctioned into the manifold from the discharge nozzle 17 side, and discharged to the outside of the case 1 through the main outlet port 22 on the front surface of the flow path manifold 20.

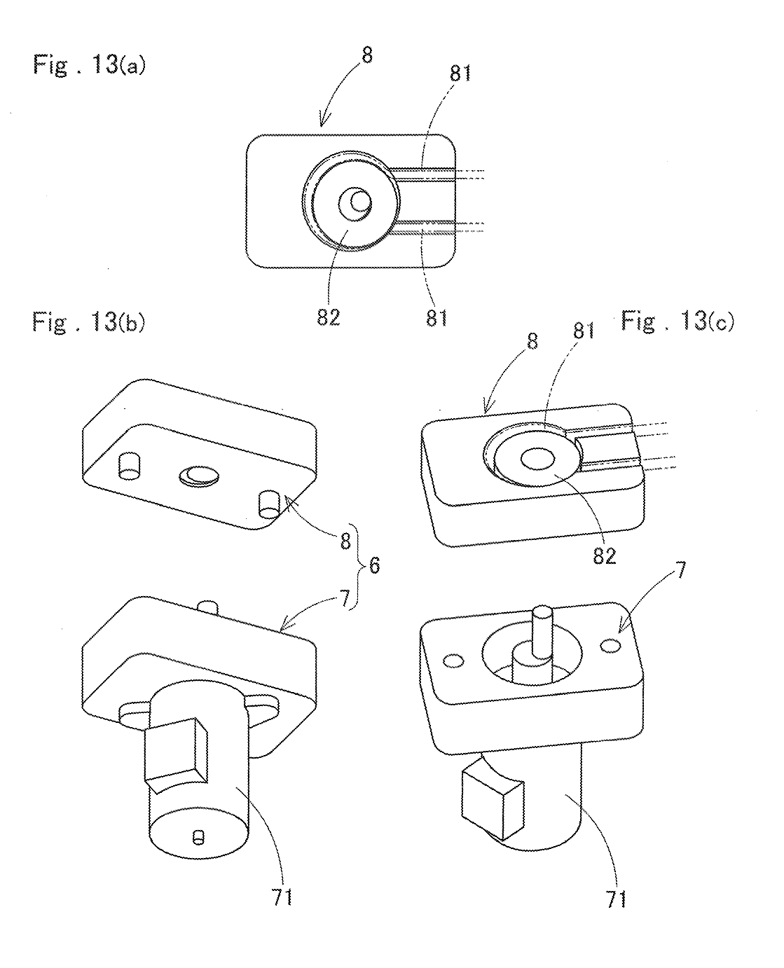

To the back surface of the flow path manifold 20, the supply liquid contact part 8 of the supply pump 6 is attached, and to the bottom surface of the flow path manifold 20, six on-off valves 18 are fixedly attached. The supply pump 6 consists of, as shown in FIG. 13, a peristaltic pump, and the supply liquid contact part 8 of the supply pump 6 includes an arc-shaped flexible flow path 81 such as a flexible tube disposed around an eccentric rotor 82 to be driven to rotate by a motor 71 of the supply pump drive part 7, and suctions a culture medium from the main inlet port 21 when the eccentric rotor 82 is driven to rotate by the motor 71. At this time, a culture medium is supplied by the supply pump 6 to flow paths of one system at a time in order, and for this, six on-off valves 18 are connected to the flow paths of the six systems.

When the supply pump 6 is driven, six on-off valves 18 are opened one by one in order, and from the six supply ports 23, a culture medium is supplied to the supply nozzle 10 side one supply nozzle at a time in order. As shown in FIG. 9, to the six supply ports 23, the supply nozzles 10 are respectively connected via the supply tubes 19, and by successive operations of the supply pump 6 and the six on-off valves 18, a culture medium is supplied from the respective supply nozzles 10 into the respective wells 4 in order.

As the on-off valve 18, a small-sized valve using, for example, a shape memory alloy for a drive part can be used. This small-sized valve is a very small valve with an orifice of approximately 0.4 mm whose power consumption is 0.3 W or less, and can be very inexpensively manufactured, and fixedly attached to the flow path manifold 20 and used as a disposable valve.

As the supply pump 6, as shown in FIG. 13, a peristaltic pump is used, however, a piezo pump having a cartridge type liquid contact part of a piezoelectric element-driven type can also be used. This cartridge type piezo pump has a structure in which a drive part of a piezoelectric element and a diaphragm chamber serving as a liquid contact part are separable, and the piezoelectric element drive part can be used as a supply pump drive part, and the diaphragm chamber can be used as a supply liquid contact part.

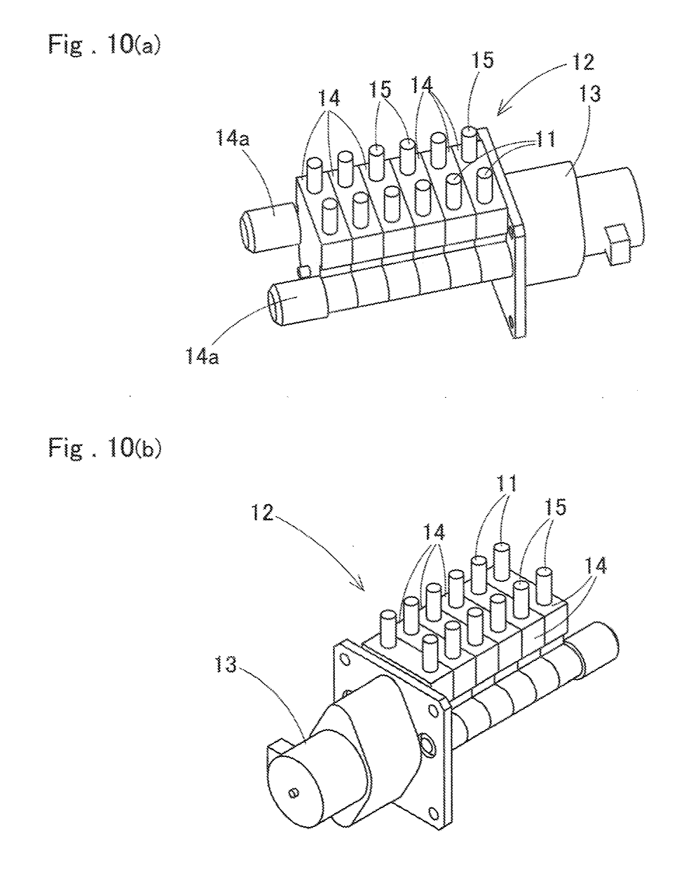

Further, to the flow path manifold 20, discharge liquid contact parts 14 of the discharge pump 12 as shown in FIG. 10 are attached to be separable from the discharge pump drive part 13, and an old culture medium is suctioned from the discharge nozzle 17 side through the six discharge ports 24 provided on the front surface, and fed from the main outlet port 22 through the branch flow paths and the discharge liquid contact parts 14 of the discharge pump 12 formed inside the manifold.

As shown in FIG. 10 to FIG. 12, the discharge pump 12 is formed as a multi-channel peristaltic pump with 6 channels by layering six discharge liquid contact parts 14, and the discharge pump drive part 13 and the discharge liquid contact parts 14 are easily separable.

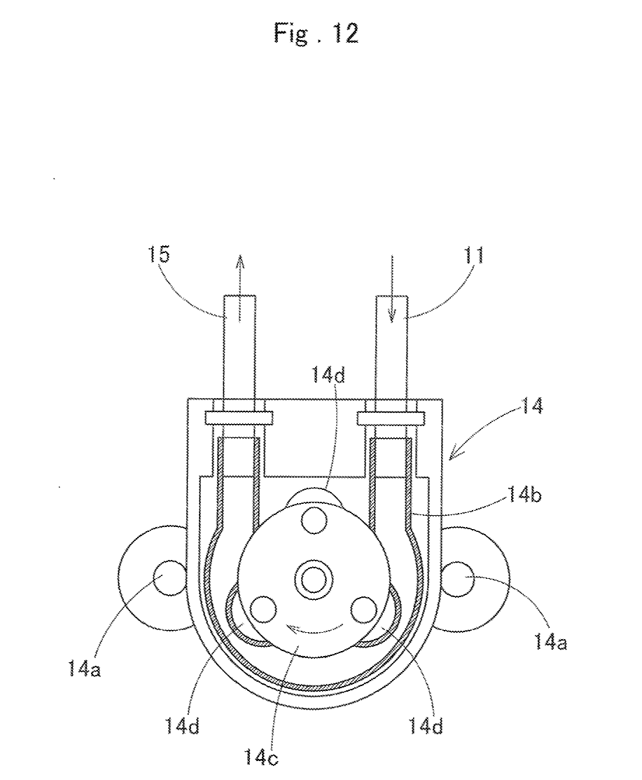

In each of the discharge liquid contact parts 14 joined in a multilayered manner, as shown in FIG. 12, a rotor 14c provided with three pressing rolls 14d inside is disposed so as to be driven to rotate, and is configured to feed a liquid by pressing an arc-shaped flexible flow path 14b being a flexible tube disposed to be contactable with each pressing roll 14d of the rotor 14c by the pressing rolls 14d in order. The discharge pump 12 rotates the rotors 14c inside the discharge liquid contact parts 14 by the discharge pump drive part 13 joined to an end portion of the discharge liquid contact parts 14 and feeds a culture medium inside the arc-shaped flexible flow paths 14b into the flow path manifold 20.

By this operation of the discharge pump 12, a culture medium is suctioned from the discharge nozzle 17 side through the six discharge ports 24 on the side surface of the flow path manifold 20, and discharged through the main outlet port 22 on the front surface of the flow path manifold 20. As shown in FIG. 9, inside the case 1, to the six discharge ports 24 of the flow path manifold 20, discharge nozzles 17 are respectively connected via the discharge tubes 11, and an old culture medium is suctioned and discharged from the respective discharge nozzles 17 inserted into the respective wells 4 of the well plate 3.

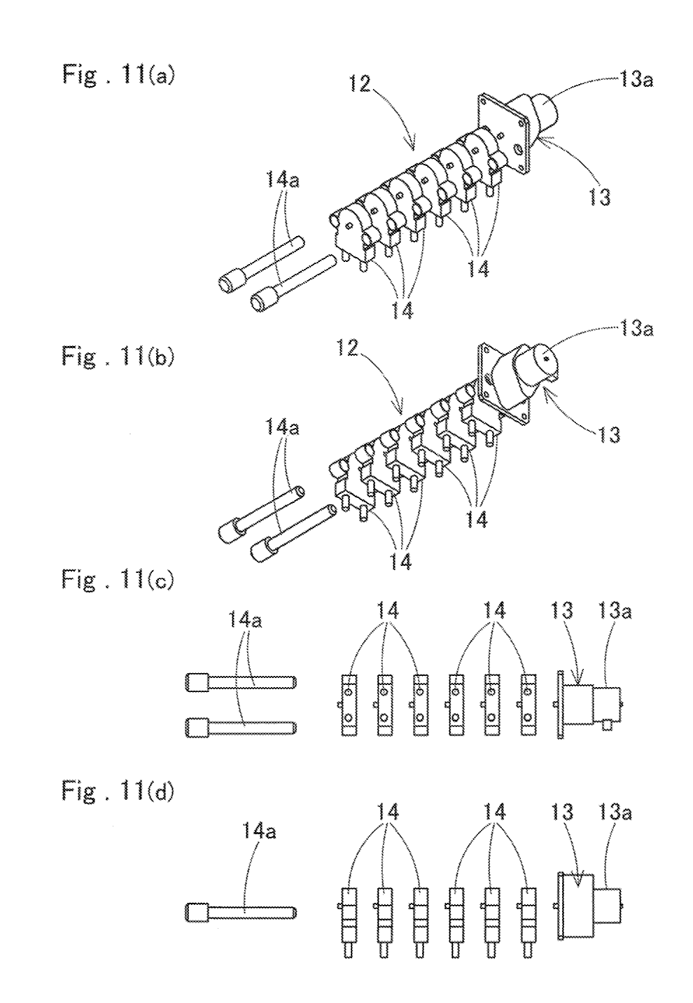

That is, the discharge pump 12 is configured by, as shown in FIG. 10 to FIG. 12, joining and fixing the discharge pump drive part 13 having a motor 13a to an end portion of the six-layered discharge liquid contact parts 14 by two fixation screws 14a. In the six discharge liquid contact parts 14, the rotors 14c each having a plurality of pressing rolls 14d are respectively axially supported so as to be driven to rotate, and a rotary shaft of the motor 13a of the discharge pump drive part 13 is joined in series to the rotors 14c so as to transmit a torque to the respective rotors 14c in order. Accordingly, the rotors inside the six discharge liquid contact parts 14 are driven to rotate by one motor 13a, and the arc-shaped flexible flow paths formed of flexible tubes disposed around the rotors are pressed in order to pump a culture medium inside the arc-shaped flexible flow paths. These six-layered discharge liquid contact parts 14 and the discharge pump drive part 13 are joined in an easily separable manner as shown in FIG. 11, and after use, by removing the two fixation screws 14a, the discharge liquid contact parts 14 can be separated from the discharge pump drive part 13 and taken out from the case 1, and cleaned or disposed of.

The flow path manifold 20 configured as described above is disposed at the lower side of the well plate 3 inside the accommodation chamber 1b of the case 1 as shown in FIG. 2, and at a lower side of the flow path manifold 20, that is, at a bottom portion in the accommodation chamber 1b, the supply pump drive part 7 for the supply pump 6 and the discharge pump drive part 13 for the discharge pump 12 are disposed by being attached to a U-shaped attaching frame 29. The supply pump drive part 7 is disposed so that its rotary shaft is turned upward, and the rotary shaft is joined to the eccentric rotor 82 as shown in FIG. 13 when the supply liquid contact part 8 is connected to cover the upper portion of the supply pump drive part 7.

The discharge pump drive part 13 is attached sideways onto the attaching frame 29, and when the flow path manifold 20 is fitted to the inside of the accommodation chamber 1b, an end portion of the discharge liquid contact parts 14 of the discharge pump 12 is connected to an end portion of the discharge pump drive part 13, and the rotary shaft of the motor 13a is joined to rotary shafts of the discharge liquid contact parts 14 as shown in FIG. 10 and FIG. 11.

Further, as described above, the main inlet port 21 and the main outlet port 22 are provided to project from the front surface of the flow path manifold 20, and on the front surface of a case corresponding to these ports, a supply opening 5 and a discharge opening 16 that are hole-shaped are provided. Accordingly, when the flow path manifold 20 is accommodated inside the accommodation chamber 1b of the case 1, the main inlet port 21 is inserted into the hole-shaped supply opening 5, the main outlet port 22 is inserted into the hole-shaped discharge opening 16, and tubes, etc., are inserted into the supply opening 5 and the discharge opening 16 from the outside. Accordingly, by connecting a tube for supply to the main inlet port 21 through the supply opening 5 and easily connecting a tube for discharge to the main outlet port 22 through the discharge opening 16, the flow path manifold 20 can be fitted to the inside of the case 1.

Adjacent to the outside of the case 1, a culture medium supply vessel to contain a fresh culture medium is installed, and a waste culture medium containing vessel to contain a discharged old waste culture medium is installed although these are not shown in the figure. The culture medium supply vessel is connected to the flow path manifold 20 through the supply opening 5 and a tube, etc., and the waste culture medium containing vessel is connected to the flow path manifold 20 through the discharge opening 16 and a tube, etc.

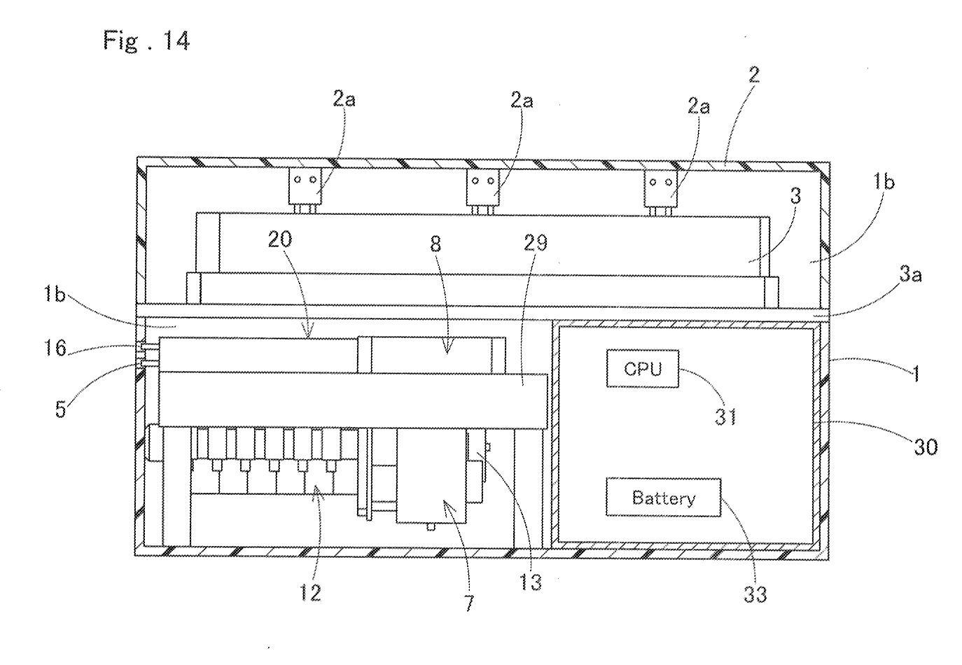

As shown in FIG. 2 and FIG. 14, in the case 1, the controller 30 that controls this culture medium exchange operation is incorporated. The controller 30 is configured by using a single-board computer including a CPU 31, and performs cell culture operation processing over time based on program data stored in advance. In the controller 30, as shown in FIG. 14, a battery 33 serving as a power source of the supply pump 6, the discharge pump 12, the on-off valves 18, and electronic circuits, etc., is incorporated, and a user interface is also provided.

In the controller 30, a culture medium supply amount that is set according to the capacity of the wells 4 of the well plate 3 is set as a time of one driving of the supply pump 6 (since a liquid feeding amount per unit time of the pump is predetermined according to pump performance), and this setting is changed each time the capacity of the wells 4 differs. In addition, in the controller 30, as a set value that a user can arbitrarily change, a culture medium exchange interval time is set, and this culture medium exchange interval time can be set on an hourly basis in a range of, for example, 6 to 18 hours. It is also allowed that the controller 30 is structured to be separately installed, and installed outside the case 1.

Next, a usage pattern of the culture medium exchange unit configured as described above is described. When performing cell culture by using this culture medium exchange unit, first, as an initial step, a predetermined amount of a culture medium (liquid culture medium) is placed into each of the wells 4 of the well plate 3, cells or body tissue to be cultured is placed into each of the wells 4, and the well plate 3 is set inside the case 1 and the opening and closing lid 2 is closed.

To the supply opening 5 in the front surface of the case 1, a culture medium supply vessel not shown in the figure is connected adjacent to the case, and to the discharge opening 16, a waste culture medium containing vessel not shown in the figure is connected, and in this state, the culture medium exchange unit is placed into an incubator controlled to a predetermined temperature, humidity, and carbon dioxide concentration, and cell culture is started.

In a case where 12 hours, for example, are set as a culture medium exchange interval time in the controller 30, for 12 hours after the start, cell culture is continued in the well plate 3 of the culture medium exchange unit. Then, when 12 hours elapses from the start, in response to a control operation of the controller 30, first, the discharge pump drive part 13 of the discharge pump 12 starts, and in each of the six discharge liquid contact parts 14 in the discharge pump 12, the rotor 14c rotates and the arc-shaped flexible flow path 14b is pressed by the pressing rolls 14d in order.

Accordingly, an old culture medium in the wells 4 is suctioned through the discharge nozzles 17, and the old culture medium enters the inside of the flow path manifold 20 from the discharge tubes 11, and further, passes through the discharge liquid contact parts 14 of the discharge pump 12, and is discharged into the waste culture medium containing vessel not shown in the figure from discharge tubes 15 and the main outlet port 22 through the discharge opening 16 in the front surface of the case 1. This old culture medium discharging operation is performed for a predetermined period of time (for example, several minutes), and accordingly, half of the old culture medium in each of the wells 4 of the well plate 3 is discharged.

Thereafter, the supply pump drive part 7 of the supply pump 6 starts, and the six on-off valves 18 successively open one by one, and a fresh culture medium suctioned from the culture medium supply vessel not shown in the figure through the supply opening 5 passes through the supply tubes 19 from the supply pump 6 and is supplied into each of the wells 4 from the respective supply nozzles 10. In the supply pump 6, the eccentric rotor 82 inside the supply liquid contact part 8 is rotated by the supply pump drive part 7, and the arc-shaped flexible flow path 81 is successively pressed by the rotation of the eccentric rotor 82, the culture medium is suctioned through the supply opening 5 and enters the flow path manifold 20 from the main inlet port 21, and the fresh culture medium is supplied into the wells 4 for a set time (by a set amount) through the supply nozzles 10 from the supply ports 23.

This supply operation is performed according to the successive operations of the six on-off valves 18, and a fresh culture medium is supplied for a set time (by a set amount) into each of the wells 4 through the respective supply nozzles 10 from the respective supply ports 23 of the flow path manifold 20. The culture medium supply amount at this time is the same as the discharged waste culture medium amount described above, and accordingly, half of the culture medium in each of the wells 4 is exchanged. The operation described above is repeated each predetermined culture medium exchange interval time, and when cell culture in the well plate 3 ends, the well plate 3 is taken out from the inside of the case 1, and a microscopic inspection, etc., of cells cultured inside the respective wells 4 are performed.

Thereafter, in the culture medium exchange unit, in preparation for the next use, the liquid contact parts are cleaned or disposed of, and new liquid contact parts are fitted to the inside of the case 1. Liquid contact parts to be taken out from the case 1 are the well plate 3, the supply nozzles 10, the discharge nozzles 17, the supply tubes 19 connected to the supply nozzles, the discharge tubes 11 connected to the discharge nozzles, and the flow path manifold 20.

As described above, to the flow path manifold 20, the supply liquid contact part 8 of the supply pump 6, the discharge liquid contact parts 14 including the discharge tubes 15 of the discharge pump 12, and the on-off valves 18 are connected, so that the flow path manifold including these liquid contact parts is taken out from the case 1 and cleaned or disposed of. The flow path manifold 20 can be easily removed in a state where the well plate 3 and the support plate 3a for the well plate are taken out from the case 1.

That is, among the supply liquid contact part 8 of the supply pump 6, the discharge liquid contact parts 14 including the discharge tubes 15 of the discharge pump 12, and the on-off valves 18, connected to the flow path manifold 20, the supply liquid contact part 8 is separated from the joint portion of the supply pump drive part 7, the discharge liquid contact parts 14 are separated from the joint portion of the discharge pump drive part 13, and the supply nozzles 10 and the discharge nozzles 17 are removed from the nozzle attaching portions 2a. Then, by removing the main inlet port 21 on the front surface of the flow path manifold 20 from the supply opening 5 in the front surface of the case 1, and removing the main outlet port 22 from the discharge opening 16, the flow path manifold 20 can be easily removed together with the supply tubes 19 and the discharge tubes 11, etc.

The removed liquid contact parts including the flow path manifold 20, etc., are cleaned or disposed of, and the cleaned liquid contact parts or new liquid contact parts are inserted into the case 1. Accordingly, cultured cells can be prevented from being contaminated.

Then, the supply liquid contact part 8 of the supply pump 6 is joined to the supply pump drive part 7, the discharge liquid contact parts 14 including the discharge tubes 15 of the discharge pump 12 are joined to the joint portion of the discharge pump drive part 13, the supply nozzles 10 and the discharge nozzles 17 are attached to the nozzle attaching portions 2a, the main inlet port 21 on the front surface of the flow path manifold 20 is connected to the external culture medium supply vessel through the supply opening 5 in the front surface of the case 1, and the main outlet port 22 is connected to the waste culture medium containing vessel through the discharge opening 16, and accordingly, with use of this culture medium exchange unit, cell culture can be performed again.

### REFERENCE SIGNS LIST

1 Case
1a Hinge
1b Accommodation chamber
2 Opening and closing lid
2a Nozzle attaching portion
3 Well plate
3a Support plate
4 Well
5 Supply opening
6 Supply pump
7 Supply pump drive part
8 Supply liquid contact part
9 Supply tube
10 Supply nozzle
11 Discharge tube
12 Discharge pump
13 Discharge pump drive part
13a Motor
14 Discharge liquid contact part
14a Fixation screw
14b Arc-shaped flexible flow path
14c Rotor
14d Pressing roll
15 Discharge tube
16 Discharge opening
17 Discharge nozzle
18 On-off valve
19 Supply tube
20 Flow path manifold
21 Main inlet port
22 Main outlet port
23 Supply port
24 Discharge port
29 Attaching frame
30 Controller
31 CPU
33 Battery
71 Motor
81 Arc-shaped flexible flow path
82 Eccentric rotor

1. A culture medium exchange unit comprising:
a case including an accommodation chamber;
a well Plate that is accommodated in the accommodation chamber of the case in a manner enabling the well plate to be taken out, and includes a plurality of wells into which cells and a culture medium are placed;
an opening and closing lid provided to cover the accommodation chamber of the case and be openable and closable;
a supply pump that supplies a culture medium into each of the wells of the well plate;
a discharge pump that discharges a used culture medium from the inside of each of the wells;
supply nozzles that supply a culture medium fed from the supply pump through supply tubes into the wells;
discharge nozzles that discharge a culture medium from the inside of the wells in response to an operation of the discharge pump;
a nozzle attaching portion that is provided inside the opening and closing lid, and holds the supply nozzles and the discharge nozzles;
a controller that controls driving of the supply pump and the discharge pump; and
a battery that serves as a power source of the supply pump, the discharge pump, and the controller, wherein
the supply pump, the discharge pump, the controller, and the battery are accommodated in the case;
to the supply pump, a supply liquid contact part serving as a culture medium flow path, and a supply pump drive part that operates to feed a culture medium into the supply liquid contact part by driving a drive part, are joined in a separable manner,
to the discharge pump, a discharge liquid contact part serving as a culture medium flow path, and a discharge pump drive part that operates to discharge a culture medium from the inside of the discharge liquid contact part by driving a drive part, are joined in a separable manner,
in the controller, a time of one driving of the supply pump and the discharge pump corresponding to a liquid feeding amount of a culture medium per unit time is set changeably, and a culture medium exchange interval time is set changeably,
supply tubes are connected to the supply nozzles, discharge tubes are connected to the discharge nozzles, and when the supply pump is driven, a culture medium is supplied into the supply nozzles from the supply liquid contact part through the supply tubes, and when the discharge pump is driven, a used culture medium is discharged from the discharge nozzles through the discharge tubes and the discharge liquid contact part,
the well plate, the supply liquid contact part of the supply pump, the supply tubes, the supply nozzles, the discharge nozzles, the discharge tubes, and the discharge liquid contact part of the discharge pump are disposed inside the case in a manner enabling them to be taken out from the case and replaced, and
cells and a culture medium are accommodated in each of the wells of the well plate, the case is placed into an incubator in which a temperature and a humidity are controlled and while cell culture is performed, the culture medium in each of the wells is exchanged by driving the supply pump and the discharge pump.

2. (canceled)

3. (canceled)

4. The culture medium exchange unit according to claim 1, wherein the supply pump consists of a peristaltic pump having an arc-shaped flexible flow path as the supply liquid contact part.

5. (canceled)

6. The culture medium exchange unit according to claim 1, wherein on the nozzle attaching portion provided inside the opening and closing lid, the supply nozzles and the discharge nozzles are held so that their tip end height positions are adjustable.

7. The culture medium exchange unit according to claim 1, wherein the discharge pump consists of a multi-channel peristaltic pump including multilayered arc-shaped flexible flow paths as the discharge liquid contact part, and the arc-shaped flexible flow paths are connected to the discharge nozzles via the discharge tubes.
